(19) United States
(12) Patent Application Publication
(10) Pub. No.: US 2024/0335664 A1
(43) Pub. Date: Oct. 10, 2024

(54) ESTIMATION SYSTEMS FOR DBS PROGRAMMING

(71) Applicant: Boston Scientific Neuromodulation Corporation, Valencia, CA (US)

(72) Inventors: Stephen Carcieri, Los Angeles, CA (US); Rafael Carbunaru, Valley Village, CA (US)

(21) Appl. No.: 18/630,224

(22) Filed: Apr. 9, 2024

Related U.S. Application Data
(60) Provisional application No. 63/458,339, filed on Apr. 10, 2023.

(51) Int. Cl. A61N 1/36; A61N 1/05; A61N 1/372; G16H 10/60; G16H 15/00; G16H 20/30; G16H 50/70

(52) U.S. Cl. CPC A61N 1/3615; A61N 1/0534; A61N 1/37241; A61N 1/37247; G16H 10/60; G16H 15/00; G16H 20/30; G16H 50/70

(57) ABSTRACT

This document discusses neurostimulation devices and methods. A neurostimulation device receives an indication of a physiological search area of a subject for a therapy mapping operation. The therapy mapping operation tests combinations of stimulation location and stimulation energy for the indicated search area. The neurostimulation device estimates one or more expected symptoms of the subject for the therapy mapping operation using symptom data for the combinations of stimulation location and stimulation energy untested during the therapy mapping operation, and presents estimations of the one or more expected symptoms for the untested combinations of stimulation location and stimulation energy.

### CLAIM OF PRIORITY

This application claims the benefit of U.S. Provisional Application No. 63/458,339 filed on Apr. 10, 2023, which is hereby incorporated by reference in its entirety.

### TECHNICAL FIELD

This document relates generally to medical devices and more particularly to systems and methods for neurostimulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Neurostimulation systems may offer many programmable options for the parameters of the neurostimulation to customize the neurostimulation therapy for a specific patient. For some types of neurostimulation (e.g., DBS) the efficacy of the neurostimulation for the patient may depend on an intricate balance of stimulation location coupled with the programmed stimulation waveform. However, the number of programmable options can create an extensive parameter search space for the physician or clinician. Finding the optimal neurostimulation parameters may take a lot of time in the clinic for both the clinic staff and the patient.

### SUMMARY

In DBS, electrical neurostimulation therapy is delivered to implantable electrodes located at certain neurostimulation targets in the brain to treat neurological or neurophysiological disorders. Device-based neurostimulation can include techniques to reduce the search space for the physician to use when customizing neurostimulation parameters to a particular patient.

Example 1 includes subject matter (machine-implemented method of controlling operation of a neurostimulation device) comprising receiving, by the neurostimulation device, an indication of a physiological search area of a subject for a therapy mapping operation, wherein the therapy mapping operation tests combinations of stimulation location and stimulation energy for the indicated search area; estimating one or more expected symptoms of the subject for the therapy mapping operation using symptom data for the combinations of stimulation location and stimulation energy untested during the therapy mapping operation; and presenting estimations of the one or more expected symptoms for the untested combinations of stimulation location and stimulation energy.

In Example 2, the subject matter of Example 1 optionally includes presenting the combinations of stimulation location and stimulation energy as points on a therapy map for the therapy mapping operation; and deriving the one or more expected symptoms for untested points on the therapy map using proximity of the untested points to tested points of the therapy map.

In Example 3, the subject matter of one or both of Examples 1 and 2 optionally includes indicating one or more symptoms of other subjects for the indicated search area for the combinations of stimulation location and stimulation energy.

In Example 4, the subject matter of Example 3 optionally includes determining a therapy map that includes points corresponding to the combinations of stimulation location and stimulation energy, and estimating the one or more expected symptoms by matching the determined therapy map to another therapy map included in a database of therapy maps.

In Example 5, the subject matter of one or both of Examples 3 and 4 optionally includes determining a therapy map that includes points corresponding to the combinations of stimulation location and stimulation energy, updating the therapy map to include tested combinations of stimulation location and stimulation energy as tested points, and uploading the therapy map to a database of therapy maps.

In Example 6, the subject matter of one or any combination of Examples 1-5 optionally includes deriving the one or more symptoms according to other physiological data of the subject.

In Example 7, the subject matter of one or any combination of Examples 1-6 optionally includes presenting the combinations of stimulation location and stimulation energy as points on a therapy map for the therapy mapping operation and presenting the one or more expected symptoms as one or more side effect volumes in the therapy map.

In Example 8, the subject matter of Example 7 optionally includes presenting one or more stimulation locations as target stimulation volumes in the therapy map.

In Example 9, the subject matter of one or both of Examples 7 and 8 optionally includes updating a side effect volume of the one or more side effect volumes in response to testing an untested point during the therapy mapping operation.

In Example 10, the subject matter of one or any combination of Examples 7-9 optionally includes presenting a confidence level of an estimation of an expected symptom of the one or more expected symptoms, testing an untested combination of a stimulation location and stimulation energy, and updating the confidence level of the estimation.

Example 11 includes subject matter (such as a neurostimulation device) or can optionally be combined with one or any combination of Examples 1-10 to include such subject matter, comprising a therapy circuit configured to deliver electrical neurostimulation energy to the one or more implantable electrodes, a port, a user interface, and a control circuit operatively coupled to the therapy circuit and the port. The control circuit is configured to receive, via the port, a selection of a physiological search area of a subject for a therapy mapping operation, wherein the control circuit initiates delivery of neurostimulation to the selected search area during the therapy mapping operation in different combinations of stimulation location and stimulation energy, receive an indication of a physiological search area for a therapy mapping operation, wherein the therapy mapping operation tests combinations of stimulation location and stimulation energy for the selected search area, estimate one or more expected symptoms of the subject for the therapy mapping operation using symptom data for the combinations of stimulation location and stimulation energy untested during the therapy mapping operation, and present, using the user interface, an indication of the one or more expected symptoms for the untested combinations of stimulation location and stimulation energy.

In Example 12, the subject matter of Example 11 optionally includes a control circuit configured to display a therapy map using the user interface, wherein the therapy map includes the combinations of stimulation location and stimulation energy as tested points and untested points on the therapy map, and derive the one or more expected symptoms for untested points on the therapy map using proximity of the untested points to the tested points of the therapy map.

In Example 13, the subject matter of one or both of Examples 11 and 12 optionally includes a control circuit configured to indicate one or more symptoms of other subjects for the selected search area for the combinations of stimulation location and stimulation energy.

In Example 14, the subject matter of one or any combination of Examples 11-13 optionally includes a memory and a control circuit configured to store a therapy map for the selected search area in the memory, the therapy map including points corresponding to the combinations of stimulation location and stimulation energy, and estimate the one or more expected symptoms by comparing the determined therapy map to another therapy map included in a database of therapy maps.

In Example 15, the subject matter of one or any combination of Examples 11-14 optionally includes a memory and a control circuit configured to store a therapy map for the selected search area in the memory, the therapy map including points corresponding to the combinations of stimulation location and stimulation energy, update the therapy map to include tested combinations of stimulation location and stimulation energy as tested points, and upload the therapy map to a database of therapy maps.

In Example 16, the subject matter of one or any combination of Examples 11-15 optionally includes a control circuit configured to display a therapy map that includes the combinations of stimulation location and stimulation energy as tested points and untested points on the therapy map, and the one or more expected symptoms displayed as one or more side effect volumes in the therapy map, initiate delivery of neurostimulation corresponding to a stimulation location and stimulation energy of an untested point of the therapy map, and update a side effect volume of the one or more side effect volumes in response to the neurostimulation.

In Example 17, the subject matter of Example 16 optionally includes a control circuit configured to display a confidence level of an estimation of an expected symptom of the one or more expected symptoms, test an untested combination of a stimulation location and stimulation energy, and update the confidence level of the estimation.

Example 18 includes subject matter (or can optionally by combined with one or any combination of Example 1-17 to include such subject matter) such as a computer readable storage medium including instructions that when performed by a control circuit of a neurostimulation device, cause the neurostimulation device to perform actions including receiving, by the neurostimulation device, an indication of a physiological search area for a therapy mapping operation to test combinations of stimulation location and stimulation energy for the indicated search area, estimating one or more expected symptoms of the subject for the therapy mapping operation using symptom data for the combinations of stimulation location and stimulation energy untested during the therapy mapping operation, and displaying estimations of the one or more expected symptoms for the untested combinations of stimulation location and stimulation energy.

In Example 19, the subject matter of Example 18 optionally includes instructions that cause the neurostimulation device to display a therapy map that includes the combinations of stimulation location and stimulation energy as tested points and untested points on the therapy map, and the one or more expected symptoms as one or more side effect volumes in the therapy map, initiate delivery of neurostimulation corresponding to a stimulation location and stimulation energy of an untested point of the therapy map, and update a side effect volume of the one or more side effect volumes in response to the neurostimulation.

In Example 20, the subject matter of one or both of Examples 18 and 19 optionally includes instructions that cause the neurostimulation device to display a confidence level of an estimation of an expected symptom of the one or more expected symptoms, initiate delivery of neurostimulation corresponding to a stimulation location and stimulation energy of an untested point of the therapy map, and update the confidence level of the estimation of the expected symptom in response to the neurostimulation.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of an electrical stimulation system.

FIGS. 2A-2C illustrate additional examples of electrical stimulation systems.

FIG. 3 is a schematic side view of an example of an electrical

stimulation lead.

FIGS. 4A-4H are illustrations of different embodiments of leads with segmented electrodes.

FIG. 5 is an illustration representing deep brain stimulation electrode implantation.

FIG. 6 is a flow diagram of an example of a method of controlling operation of a neurostimulation device.

FIG. 7 is a block diagram of an example of portions of a neurostimulation device.

FIG. 8 is an example of a therapy map.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS therapy.

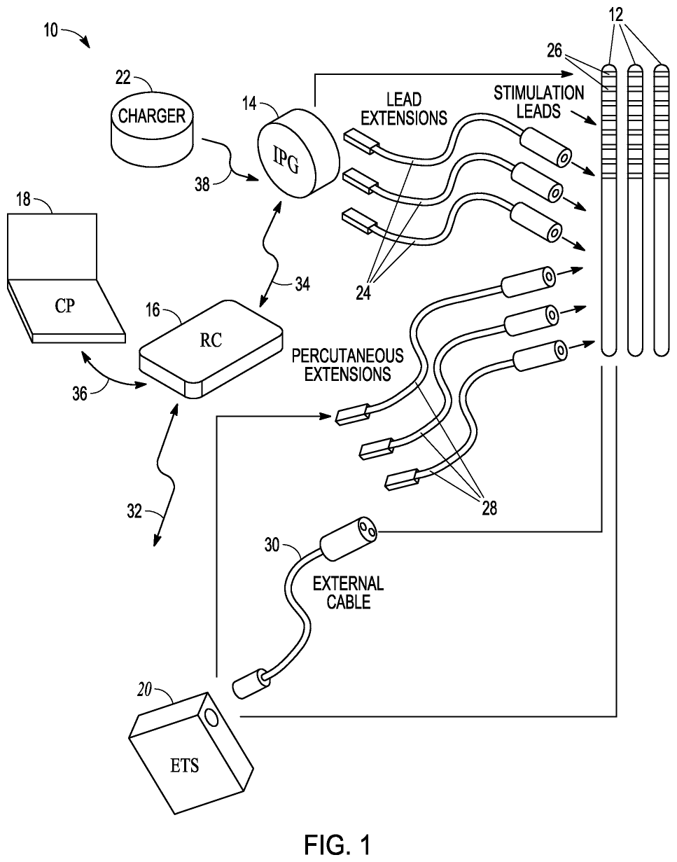

FIG. 1 is an illustration of portions of an embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

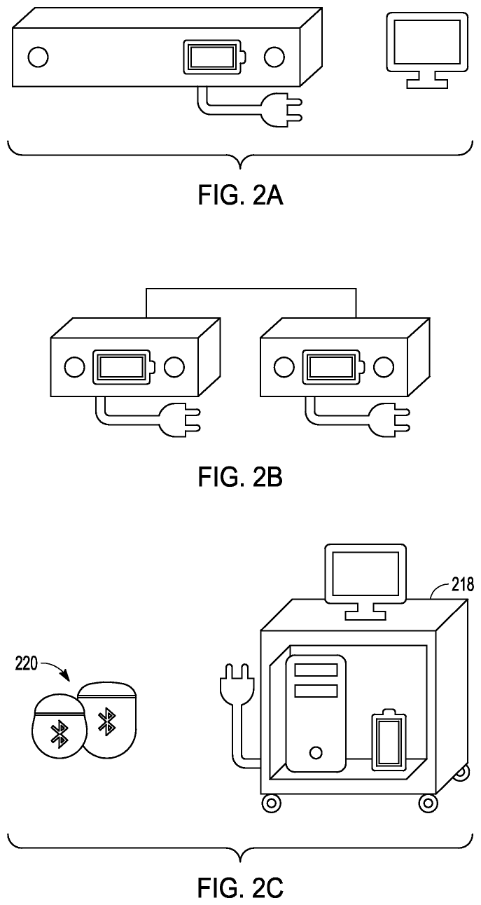

FIGS. 2A-2C illustrate examples of additional hardware configurations. In FIG. 2A, the electrical stimulation hardware, the sensing hardware, and a user interface are included in the same unit. In FIG. 2B, the electrical stimulation hardware and the sensing hardware are provided as separate units. The unit or units of FIGS. 2A and 2B can be capital equipment and can be rack mounted. The units shown in FIGS. 2A and 2B are wall powered but the units can be battery powered. FIG. 2C illustrates an example that includes an ETS 220 and separate computer 218. The computer 218 communicates wirelessly with the ETS 220 (e.g., using Bluetooth hardware and communication protocol).

Returning to FIG. 1, the RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is incorporated herein by reference. Other embodiments of electrical stimulation systems can be found at U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; 8,364,278; and 8,700,178, all of which are incorporated herein by reference.

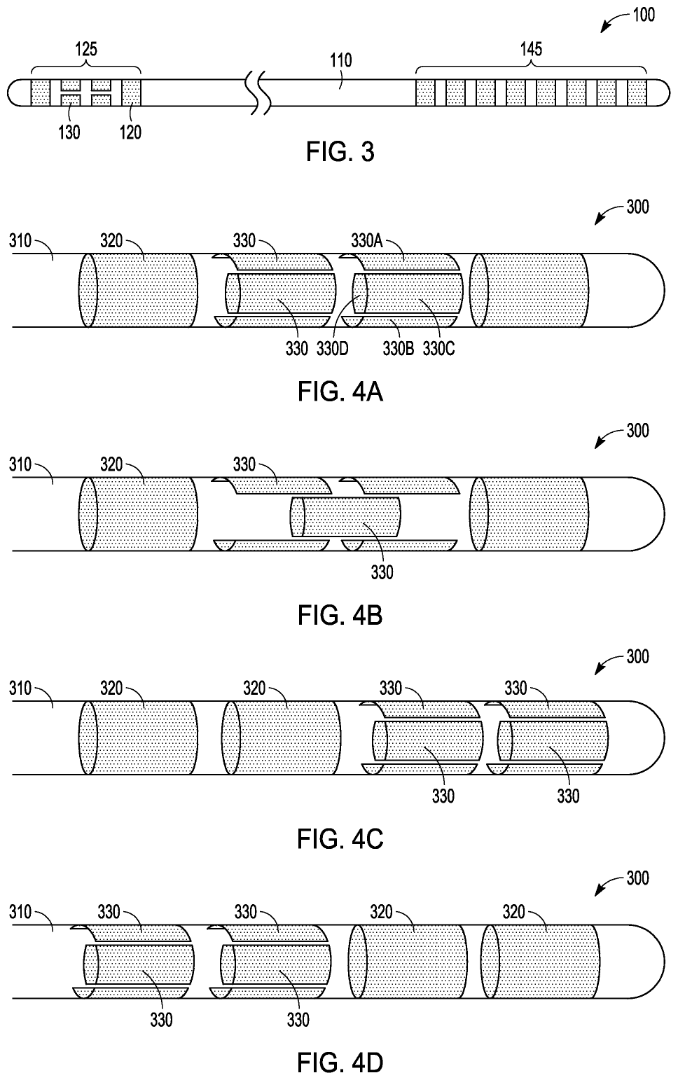

FIG. 3 is a schematic side view of an embodiment of an electrical stimulation lead. FIG. 3 illustrates a lead 110 with electrodes 125 disposed at least partially about a circumference of the lead 110 along a distal end portion of the lead and terminals 145 disposed along a proximal end portion of the lead. The electrodes can be used to spatially distribute fields of neurostimulation energy. The lead 110 can be implanted near or within the desired portion of the body to be stimulated (e.g., the brain, spinal cord, or other body organs or tissues). In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 110 can be inserted into the cranium and brain tissue with the assistance of a stylet (not shown). The lead 110 can be guided to the target location within the brain using, for example, a stereotactic frame and a microdrive motor system, optionally including cannulas. In some embodiments, the microdrive motor system can be fully or partially automatic. The microdrive motor system may be configured to perform one or more the following actions (alone or in combination): insert the lead 110, advance the lead 110, retract the lead 110, or rotate the lead 110.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 110 for deep brain stimulation can include stimulation electrodes, recording electrodes, or both. In at least some embodiments, the lead 110 is rotatable so that the stimulation electrodes can be aligned with the target neurons after the neurons have been located using the recording electrodes. Stimulation electrodes may be disposed on the circumference of the lead 110 to stimulate the target neurons. Stimulation electrodes may be ring-shaped so that current projects from each electrode equally in every direction from the position of the electrode along a length of the lead 110. In the embodiment of FIG. 3, two of the electrodes are ring electrodes 120, 122. Ring electrodes typically do not enable stimulus current to be directed from only a limited angular range around of the lead. Segmented electrodes 130, 132, however, can be used to direct stimulus current to a selected angular range around the lead. When segmented electrodes are used in conjunction with an implantable pulse generator that delivers constant current stimulus with more than one independent stimulus source, current steering can be achieved to more precisely deliver the stimulus to a position along and around an axis of the lead (e.g., longitudinal and radial positioning around the axis of the lead). When current steering, segmented electrodes can be utilized in addition to, or as an alternative to, ring electrodes.

The lead 100 includes a lead body 110, terminals 145, and one or more ring electrodes 120, 122 and one or more sets of segmented electrodes 130, 132 (or any other combination of electrodes). The lead body 110 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 100 may be in contact with body tissue for extended periods of time. In at least some embodiments, the lead 100 has a cross-sectional diameter of no more than 1.5 millimeters (1.5 mm) and may be in the range of 0.5 to 1.5 mm. In at least some embodiments, the lead 100 has a length of at least 10 centimeters (10 cm) and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads and other leads may include one or more sets of segmented electrodes. Segmented electrodes may provide for finer current steering than ring electrodes because target structures in deep brain stimulation or other stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other non-target tissue.

Any number of segmented electrodes 130, 132 may be disposed on the lead body 110 including, for example, anywhere from one to sixteen or more segmented electrodes. It will be understood that any number of segmented electrodes may be disposed along the length of the lead body 110. A segmented electrode 130, 132 typically extends only 75%, 67%, 60%, 50%, 40%, 33%, 25%, 20%, 17%, 15%, or less around the circumference of the lead.

The segmented electrodes may be grouped into sets of segmented electrodes, where each set is disposed around a circumference of the lead 100 at a particular longitudinal portion of the lead 100. The lead 100 may have any number segmented electrodes in a given set of segmented electrodes. The lead 100 may have one, two, three, four, five, six, seven, eight, or more segmented electrodes in a given set. In at least some embodiments, each set of segmented electrodes of the lead 100 contains the same number of segmented electrodes. The segmented electrodes disposed on the lead 100 may include a different number of electrodes than at least one other set of segmented electrodes disposed on the lead 100. The segmented electrodes may vary in size and shape. In some embodiments, the segmented electrodes are all of the same size, shape, diameter, width or area or any combination thereof. In some embodiments, the segmented electrodes of each circumferential set (or even all segmented electrodes disposed on the lead 100) may be identical in size and shape.

Each set of segmented electrodes may be disposed around the circumference of the lead body 110 to form a substantially cylindrical shape around the lead body 110. The spacing between individual electrodes of a given set of the segmented electrodes may be the same, or different from, the spacing between individual electrodes of another set of segmented electrodes on the lead 100. In at least some embodiments, equal spaces, gaps or cutouts are disposed between each segmented electrode around the circumference of the lead body 110. In other embodiments, the spaces, gaps or cutouts between the segmented electrodes may differ in size, or cutouts between segmented electrodes may be uniform for a particular set of the segmented electrodes or for all sets of the segmented electrodes. The sets of segmented electrodes may be positioned in irregular or regular intervals along a length the lead body 110.

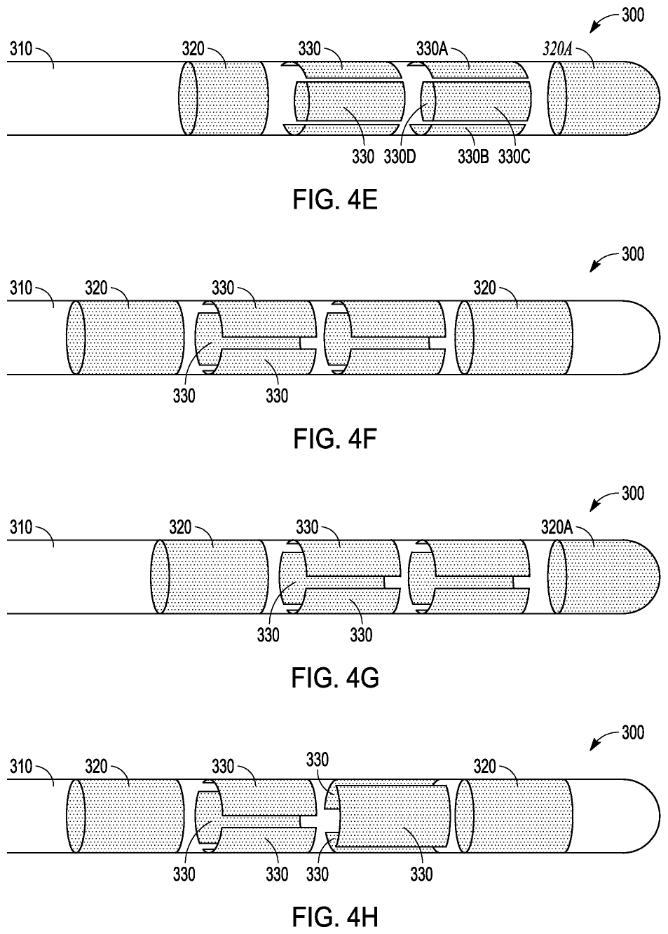

Conductor wires (not shown) that attach to the ring electrodes 120, 122 or segmented electrodes 130, 132 extend along the lead body 110. These conductor wires may extend through the material of the lead 100 or along one or more lumens defined by the lead 100, or both. The conductor wires couple the electrodes 120, 122, 130, 132 to the terminals 145. FIGS. 4A-4H are illustrations of different embodiments of leads 300 with segmented electrodes 330, optional ring electrodes 320 or tip electrodes 320a, and a lead body 310. The sets of segmented electrodes 330 each include either two (FIG. 4B), three (FIGS. 4E-4H), or four (FIGS. 4A, 4C, and 4D) or any other number of segmented electrodes including, for example, three, five, six, or more. The sets of segmented electrodes 330 can be aligned with each other (FIGS. 4A-4G) or staggered (FIG. 4H).

When the lead 100 includes both ring electrodes 120, 122 and segmented electrodes 130, 132, the ring electrodes and the segmented electrodes may be arranged in any suitable configuration. For example, when the lead 100 includes two ring electrodes and two sets of segmented electrodes, the ring electrodes can flank the two sets of segmented electrodes (see e.g., FIGS. 3, 4A, and 4E-4H, ring electrodes 320 and segmented electrode 330). Alternately, the two sets of ring electrodes can be disposed proximal to the two sets of segmented electrodes (see e.g., FIG. 4C, ring electrodes 320 and segmented electrode 330), or the two sets of ring electrodes can be disposed distal to the two sets of segmented electrodes (see e.g., FIG. 4D, ring electrodes 320 and segmented electrode 330). One of the ring electrodes can be a tip electrode (see e.g., tip electrode 320a of FIGS. 4E and 4G). It will be understood that other configurations are possible as well (e.g., alternating ring and segmented electrodes, or the like).

By varying the location of the segmented electrodes, different coverage of the target neurons may be selected. For example, the electrode arrangement of FIG. 4C may be useful if the physician anticipates that the neural target will be closer to a distal tip of the lead body 110, while the electrode arrangement of FIG. 4D may be useful if the physician anticipates that the neural target will be closer to a proximal end of the lead body 110.

Any combination of ring electrodes and segmented electrodes may be disposed on the lead 100. For example, the lead may include a first ring electrode, two sets of segmented electrodes; each set formed of four segmented electrodes, and a final ring electrode at the end of the lead. This configuration may simply be referred to as a 1-4-4-1 configuration (FIGS. 4A and 4E, ring electrodes 320 and segmented electrode 330). It may be useful to refer to the electrodes with this shorthand notation. Thus, the embodiment of FIG. 4C may be referred to as a 1-1-4-4 configuration, while the embodiment of FIG. 4D may be referred to as a 4-4-1-1 configuration. The embodiments of FIGS. 4F, 4G, and 4H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes are disposed on the lead. The 1-3-3-1 electrode configuration of FIGS. 4F, 4G, and 4H has two sets of segmented electrodes, each set containing three electrodes disposed around the circumference of the lead, flanked by two ring electrodes (FIGS. 4F and 4H) or a ring electrode and a tip electrode (FIG. 4G). In some embodiments, the lead includes 16 electrodes. Possible configurations for a 16-electrode lead include, but are not limited to 4-4-4-4; 8-8; 3-3-3-3-3-1 (and all rearrangements of this configuration); and 2-2-2-2-2-2-2-2

Any other suitable arrangements of segmented and/or ring electrodes can be used including, but not limited to, those disclosed in U.S. Patent Applications Publication Nos. 2012/0197375, 2015/0045864, and 2016/0228692, which are incorporated herein by reference. As an example, arrangements in which segmented electrodes are arranged helically with respect to each other. One embodiment includes a double helix. The leads and electrodes described in regarding to FIG. 1 through 4H are some examples of the types of leads and electrodes that can be used to implement the techniques described herein. Other arrangements of leads and electrodes can also be used.

One or more electrical stimulation leads can be implanted in the body of a patient and used to stimulate surrounding tissue. The electrical stimulation leads can provide electrical neurostimulation to multiple stimulation sites after implantation and to provide different stimulation geometries to the patient.

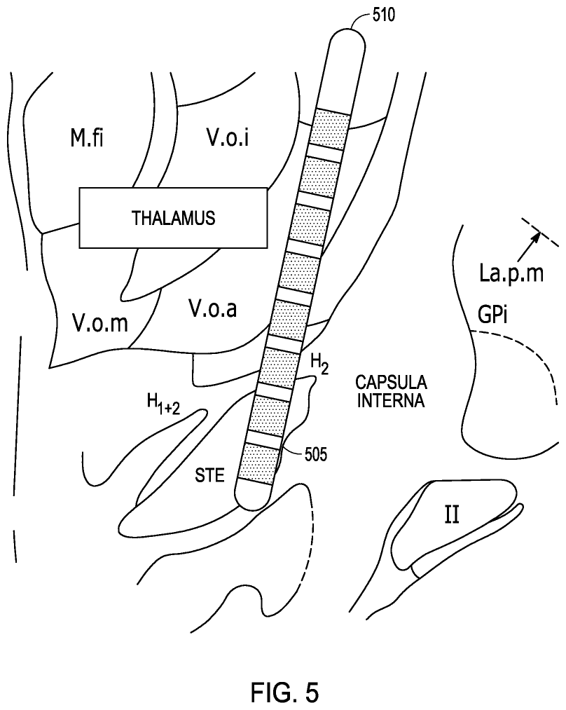

FIG. 5 is an illustration representing DBS electrode implantation on or near the subthalamic nucleus (STN) 505. The DBS electrodes of lead 510 are connected to a therapy circuit of an IPG 14 or ETS 20. DBS lead(s) are coupled to the implantable pulse generator (such as IPG 14 in FIG. 1). When the DBS lead(s) are implanted, the lead(s) can be connected to an ETS and neurostimulation can be delivered to the patient using the ETS. The ETS can be used to test the efficacy of the position of the DBS lead(s). After implantation, a clinician will program the IPG 14 using the clinician programmer, remote control, or other programming device. According to at least some programming techniques, the clinician enters stimulator parameters for a stimulation program and the stimulation program is used to stimulate the patient. The clinician observes the patient response. In at least some instances, the clinician asks the patient to describe, rate, or otherwise provide information about the effects of the stimulation such as how strong is the stimulation effect is, whether there are side effects or negative effects, and the like.

As explained previously herein, some modes of neurostimulation (such as DBS) may depend on an intricate balance of stimulation location with the correct stimulation waveform. Neurostimulation systems can be programmable in stimulation sites, stimulation electrode combinations, stimulation pulse amplitude, pulse width, pulse rate, and pulse pattern to provide many different neurostimulation waveforms. Programming to find the best combination of stimulation parameters for DBS can often be slow and labor intensive. This is becoming especially true as new systems are developed that include capabilities that expand the number of electrodes available for stimulation, provide directional stimulation steering, and provide wider stimulation parameter ranges.

An improved approach would automatically narrow down the search space for spatiotemporal settings for neurostimulation based on a prediction of the symptoms resulting from the settings. The confidence in the predicted results can be estimated to present settings with the highest confidence to the clinician.

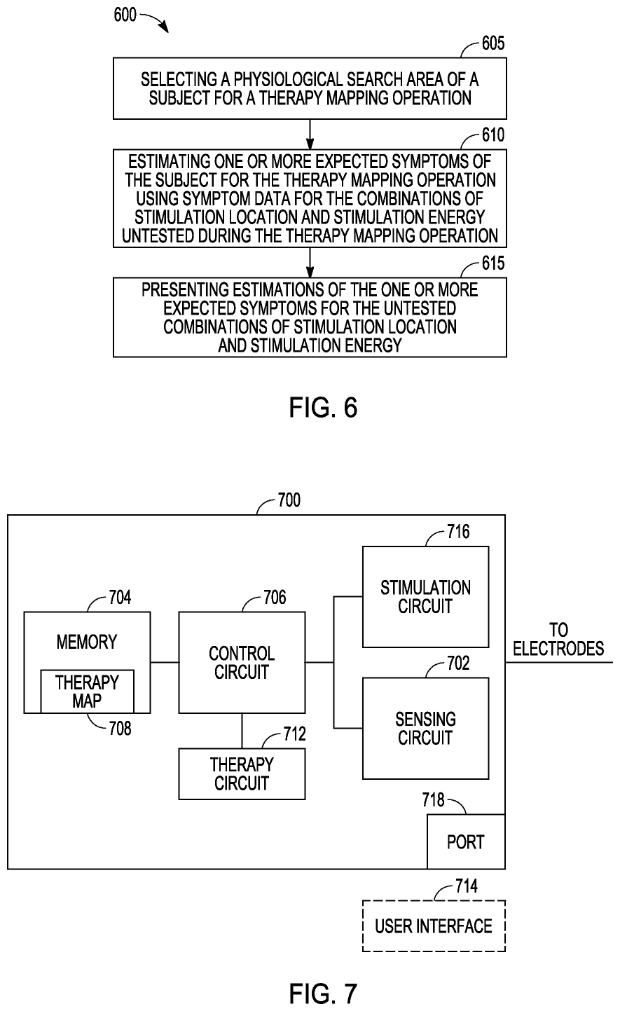

FIG. 6 is a flow diagram of an example of a method 600 of controlling operation of a neurostimulation device. At block 605, a user (e.g., a clinician) selects a physiological search area of a patient or subject for a therapy mapping operation. The therapy mapping operation includes delivering electrical neurostimulation to test combinations of stimulation location and stimulation energy for the indicated search area. In some examples, the user enters the search area using a user interface of the neurostimulation device. The user interface can include one or more of a mouse, a keyboard, and a display (e.g., a touchscreen display). In some examples, the search area is indicated by the positions of one or more DBS leads (e.g., DBS lead 510 in the example of FIG. 5) when the DBS lead(s) are implanted.

At block 610, the neurostimulation device estimates or predicts one or more expected symptoms of the subject for the therapy mapping operation using symptom data for the combinations of stimulation location and stimulation energy untested during the therapy mapping operation. The symptoms may be symptoms related to the physiological condition of the subject to be treated (e.g., Parkinsonian symptoms) or the symptoms may be side effects of negative effects of the neurostimulation. The symptoms are estimated or predicted because the prediction is for neurostimulation that has not been tested yet during the therapy mapping operation. At block 615, the expected symptoms are presented to the user (e.g., using a display of the neurostimulation device).

FIG. 7 is a block diagram of an example of portions of a neurostimulation device 700 that predicts symptoms of the subject that will result from neurostimulation delivered using the device before the neurostimulation is selected and delivered. The neurostimulation device 700 may be an ETS 20. The neurostimulation device 700 includes a stimulation circuit 716, a sensing circuit 702, a memory 704, and a control circuit 706 in communication with the stimulation circuit 716, the sensing circuit 702, and the memory 704. The neurostimulation device 700 may include a user interface 714. The neurostimulation device 700 may include a port 718 to receive information from a separate device or from the user interface 714. The port 718 may be a communication port (COMM Port) that can ne a serial port (e.g., universal serial bus (USB) port) or parallel port. The port 718 may be connected to a communication circuit to receive information wirelessly from a second device. The sensing circuit 702 may include one or more sense amplifiers that sense neural signals of a subject when the sensing circuit 702 is connected to one or more implantable electrodes (e.g., electrodes of an implantable DBS lead). In some examples, the implantable electrodes are configured by shape and size for placement on or near a deep brain structure such as a basal ganglion of the brain or the STN of the brain.

The stimulation circuit 716 delivers electrical neurostimulation when the stimulation circuit 716 is connected to the implantable electrodes. The control circuit 706 can include processing circuitry such as a microprocessor, a digital signal processor, application specific integrated circuit (ASIC), or other type of processor, interpreting or executing instructions in software or firmware stored in the memory of the device. The memory 704 is connected to, or integral to, the control circuit 706.

The control circuit 706 initiates delivery of neurostimulation to perform a therapy mapping operation for a patient. For a therapy mapping operation, the physiological search area for the mapping is input to the neurostimulation device 700. The search area may be input via the user interface 714, or the search area may be received from a separate device (e.g., a separate medical device used to place the implantable DBS electrodes). The therapy mapping operation involves the control circuit 706 initiating delivery of neurostimulation to combinations of stimulation location and stimulation energy. The stimulation location is determined by the placement of the lead and the orientation of the electrodes. The stimulation location may be changed by the control circuit 706 changing the electrodes or segments of electrodes used to provide the stimulation to the search area. The stimulation energy can be changed by the control circuit 706 changing one or more of the stimulation pulse amplitude, pulse width, pulse rate, and pulse pattern.

The control circuit 706 executes instructions that implement an algorithm to estimate the symptoms that will result from the neurostimulation options available to the therapy mapping operation. The estimated symptoms are presented to the user as an aid in selecting the combinations of stimulation location and stimulation energy for the mapping.

In some examples, the control circuit 706 generates a therapy map 708 that includes points corresponding to the combinations of stimulation location and stimulation energy. The therapy map 708 may be stored in memory 704.

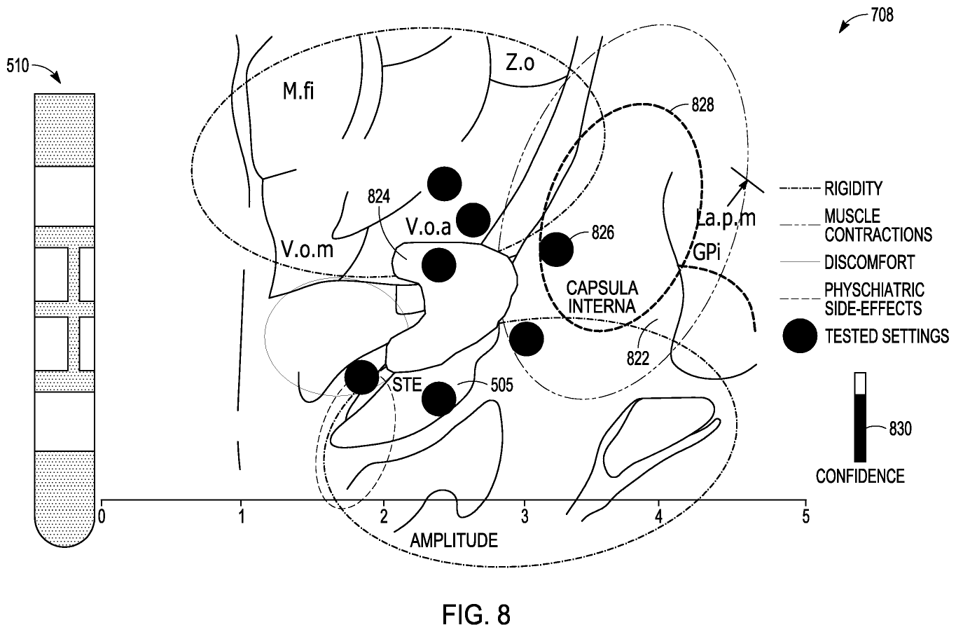

FIG. 8 is an example of a therapy map 708 that may be presented to user on a display of the neurostimulation device or the neurostimulation device may send information of the therapy map to another device or display. On the vertical axis is a representation of electrode positions of a DBS lead 510. Selection of one or a combination of electrodes is associated to the stimulation location. The horizontal axis includes values of stimulation amplitude (e.g., in Volts). The therapy map associates electrode position to stimulation amplitude. Thus, the therapy map is a graph of points corresponding to combinations of stimulation location and stimulation amplitude.

The therapy map shows seven points that were tested, and symptom data is available for the patient for those tested points. The therapy map 708 is shown superimposed on anatomical features of the brain such as the STN 505 and the capsula interna 822. The therapy map 708 also shows the physiological search area 824 selected by the user as a target stimulation volume. The therapy map 708 also presents expected symptoms as side effect volumes in the therapy map. The therapy map 708 example shown includes side effect volumes for rigidity, muscle contractions, discomfort and psychiatric side effects. The side effect volumes are the predicted side effects for those untested neurostimulation points that are the combinations of stimulation location and stimulation energy within those volumes. In variations, the therapy map 708 only display one of the vertical axis or horizontal axis, and the therapy map 708 associates the physiological search and side effect volumes to only one of the stimulation location or the stimulation energy.

According to some examples, the expected symptoms for untested points on the therapy map are derived by the control circuit 706 using proximity of the untested points to tested points of the therapy map. For example, stimulation according to tested point 826 may have resulted in muscle contractions of the patient. Based on proximity to the tested point 826, the control circuit 706 identifies side effect volume 828 as a volume of points where neurostimulation corresponding to the untested points is likely to cause muscle contractions. The side effect volume 828 is presented as a heat map, with darker concentration indicating a higher likelihood of causing the side effect.

In some examples, the expected symptoms for the untested points are derived from those symptoms that are expected based on other physiological data of the patient. For example, the control circuit 706 may have access to anatomical data of the patient, or electrophysiological data for the patient. Reading the data for the patient undergoing the mapping operation can allow the control circuit 706 to predict the symptoms of the untested points of the therapy map 708.

In some examples, the control circuit 706 derives the expected symptoms for the untested points from those symptoms directly measured for tested points. The symptoms may be detected by a device separate from the neurostimulation device 700. For example, a wearable device or an external sensor (e.g., an external accelerometer) may detect muscle contractions of the patient at one or more tested points. The detection by the separate device may be stored with data that is accessible by the neurostimulation device 700 or the detection may be communicated wirelessly from the separate device to the neurostimulation device 700. The control circuit 706 may predict that the symptoms of the untested point will be the same as for the tested point based on proximity of the untested point to the tested point.

In some examples, the control circuit 706 can access a database of therapy maps. The database may be stored in the memory 704 and may include therapy maps from previous therapy mapping operations of a patient population. In certain examples, the therapy map database is a cloud database accessible by the control circuit 706. The control circuit 706 determines the expected symptoms of the patient by matching the current therapy map to therapy maps of other patients in the database. For example, the therapy maps may be categorized by lead location, such as therapy maps for a lead location near the STN 505 as in FIG. 8. The control circuit 706 may identify therapy maps in the database that have similar lead location and a similar physiological search area 824. If the identified therapy maps are for patients with a similar disease for the current patient, the control circuit may add symptoms to the therapy map based on the symptoms and side effects for the current patient using the database therapy maps of the other patients.

With the therapy map 708 displayed for the clinician, the clinician may choose untested points of the therapy map 708 for testing during the therapy mapping operation. When a new point on the therapy map 708 is tested, the control circuit 706 may update a side effect volume of the therapy map in response to the testing. For example, if testing a point near the muscle contraction side effect volume 828 produces a muscle contraction, the control circuit 706 may expand the side effect volume 828 in response to the neurostimulation results. The tested point is added to the therapy may as a tested point. At the end of the therapy mapping operation, the control circuit 706 may store the therapy map resulting from the operation in the database or upload the new therapy map to a cloud database to register the new therapy map in the database.

In some examples, the control circuit computes a confidence level for the predicted symptoms of the therapy map. The confidence may be determined based on the method used to derive the predicted symptom. For example, if multiple therapy maps were identified in the database that were similar to the current patient map in stimulation points and symptoms, the control circuit 706 would determine that the confidence level is high that the predicted symptoms of the current map were correct.

The control circuit 706 may present the confidence level of the predictions on the therapy map such as by a confidence status bar 830. In certain examples, the control circuit 706 may highlight regions of the therapy map as having higher confidence of the predictions. The user may select a range of settings in the high confidence range for the therapy mapping operation to reduce the range of points to be tested during the mapping operation. Alternatively, the user may select a range of points in a lower confidence region to improve the accuracy of the estimates in that region.

The control circuit 706 may display to the user recommended specific settings to test during the therapy mapping operation. These settings may be determined to reduce the uncertainty in the maps or may be designed to perform a comparison between estimated symptoms and measured symptoms and determine the error in the estimates. The results from these settings can be used to update the therapy map 708 to reduce error in the predictions.

In some examples, the control circuit 706 executes instructions that implement an algorithm to optimize stimulation parameters once a stimulation location is selected according to different metrics. For example, if the neurostimulation device determines that different combinations of pulse width and amplitude provide a similar volume of stimulation, the algorithm recommends a combination of pulse width and amplitude that consumes less stimulation energy, or a combination that has a larger therapeutic window.

The devices, systems and methods described herein provide techniques for neurostimulation effects mapping during DBS that estimate the symptoms at locations of the map that have not yet been tested for the mapping operation. The techniques described allow symptoms of all points of the therapy map to estimated based on the results of a small number of points actually tested.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A machine-implemented method of controlling operation of a neurostimulation device, the method comprising:
receiving, by the neurostimulation device, an indication of a physiological search area of a subject for a therapy mapping operation, wherein the therapy mapping operation tests one or both of stimulation location and stimulation energy for the indicated search area;
estimating one or more expected symptoms of the subject for the therapy mapping operation using symptom data for the one or both of stimulation location and stimulation energy untested during the therapy mapping operation; and
presenting estimations of the one or more expected symptoms for one or both of the untested stimulation location and the untested stimulation energy.

2. The method of claim 1, including:
presenting, using a display of the neurostimulation device, combinations of stimulation location and stimulation energy as points on a therapy map for the therapy mapping operation; and
wherein estimating the one or more expected symptoms includes deriving the one or more expected symptoms for untested points on the therapy map using proximity of the untested points to tested points of the therapy map.

3. The method of claim 2, wherein estimating the one or more expected symptoms includes indicating one or more symptoms of other subjects for the indicated search area for the combinations of stimulation location and stimulation energy.

4. The method of claim 3, including:
determining a therapy map that includes points corresponding to the combinations of stimulation location and stimulation energy; and
wherein estimating the one or more expected symptoms includes estimating the one or more expected symptoms by matching the determined therapy map to another therapy map included in a database of therapy maps.

5. The method of claim 3, including:
determining a therapy map that includes points corresponding to the combinations of stimulation location and stimulation energy;
updating the therapy map to include tested combinations of stimulation location and stimulation energy as tested points; and
uploading the therapy map to a database of therapy maps.

6. The method of claim 2, wherein estimating the one or more expected symptoms includes deriving the one or more symptoms according to other physiological data of the subject.

7. The method of claim 2, including:
presenting, using a display of the neurostimulation device, the combinations of stimulation location and stimulation energy as points on a therapy map for the therapy mapping operation; and
presenting the one or more expected symptoms as one or more side effect volumes in the therapy map.

8. The method of claim 7, including presenting one or more stimulation locations as target stimulation volumes in the therapy map.

9. The method of claim 7, including updating a side effect volume of the one or more side effect volumes in response to testing an untested point during the therapy mapping operation.

10. The method of claim 7, including:
presenting a confidence level of an estimation of an expected symptom of the one or more expected symptoms;
testing an untested combination of a stimulation location and stimulation energy; and
updating the confidence level of the estimation.

11. A neurostimulation device for electrical connection to one or more implantable electrodes, the neurostimulation device comprising:
a therapy circuit configured to deliver electrical neurostimulation energy to the one or more implantable electrodes;
a port;
a user interface; and
a control circuit operatively coupled to the therapy circuit and the port and configured to:
receive, via the port, a selection of a physiological search area of a subject for a therapy mapping operation, wherein the control circuit initiates delivery of neurostimulation to the selected search area during the therapy mapping operation in different combinations of stimulation location and stimulation energy;
receive an indication of a physiological search area for a therapy mapping operation, wherein the therapy mapping operation tests one or both of stimulation location and stimulation energy for the selected search area;
estimate one or more expected symptoms of the subject for the therapy mapping operation using symptom data for the one or both of stimulation location and stimulation energy untested during the therapy mapping operation; and
present, using the user interface, an indication of the one or more expected symptoms for one or both of the untested stimulation location and the untested stimulation energy.

12. The neurostimulation device of claim 11, wherein the control circuit is configured to:
display a therapy map using the user interface, wherein the therapy map includes combinations of stimulation location and stimulation energy as tested points and untested points on the therapy map; and
derive the one or more expected symptoms for untested points on the therapy map using proximity of the untested points to the tested points of the therapy map.

13. The neurostimulation device of claim 12, wherein the control circuit is configured to indicate one or more symptoms of other subjects for the selected search area for the combinations of stimulation location and stimulation energy.

14. The neurostimulation device of claim 12, including:
a memory operatively coupled to, or integral to, the control circuit;
wherein the control circuit is configured to:
store a therapy map for the selected search area in the memory, the therapy map including points corresponding to the combinations of stimulation location and stimulation energy; and
estimate the one or more expected symptoms by comparing the determined therapy map to another therapy map included in a database of therapy maps.

15. The neurostimulation device of claim 12, including:
a memory operatively coupled to, or integral to, the control circuit;
wherein the control circuit is configured to:
store a therapy map for the selected search area in the memory, the therapy map including points corresponding to the combinations of stimulation location and stimulation energy;
update the therapy map to include tested combinations of stimulation location and stimulation energy as tested points; and
upload the therapy map to a database of therapy maps.

16. The neurostimulation device of claim 12, wherein the control circuit is configured to:
display a therapy map using the user interface, wherein the therapy map includes the combinations of stimulation location and stimulation energy as tested points and untested points on the therapy map, and the one or more expected symptoms displayed as one or more side effect volumes in the therapy map;
initiate delivery of neurostimulation corresponding to a stimulation location and stimulation energy of an untested point of the therapy map; and
update a side effect volume of the one or more side effect volumes in response to the neurostimulation.

17. The neurostimulation device of claim 16, wherein the control circuit is configured to:
display a confidence level of an estimation of an expected symptom of the one or more expected symptoms;
test an untested combination of a stimulation location and stimulation energy; and
update the confidence level of the estimation.

18. A non-transitory computer readable storage medium including instructions that when performed by a control circuit of a neurostimulation device, cause the neurostimulation device to perform actions including:
receiving, by the neurostimulation device, an indication of a physiological search area for a therapy mapping operation, wherein the therapy mapping operation tests combinations of stimulation location and stimulation energy for the indicated search area;
estimating one or more expected symptoms of the subject for the therapy mapping operation using symptom data for the combinations of stimulation location and stimulation energy untested during the therapy mapping operation; and
displaying estimations of the one or more expected symptoms for the untested combinations of stimulation location and stimulation energy.

19. The non-transitory computer-readable storage medium of claim 18, including instructions that cause the neurostimulation device to:
display a therapy map that includes the combinations of stimulation location and stimulation energy as tested points and untested points on the therapy map, and the one or more expected symptoms as one or more side effect volumes in the therapy map;
initiate delivery of neurostimulation corresponding to a stimulation location and stimulation energy of an untested point of the therapy map; and
update a side effect volume of the one or more side effect volumes in response to the neurostimulation.

20. The non-transitory computer-readable storage medium of claim 18, including instructions that cause the neurostimulation device to:
display a confidence level of an estimation of an expected symptom of the one or more expected symptoms;
initiate delivery of neurostimulation corresponding to a stimulation location and stimulation energy of an untested point of the therapy map; and
update the confidence level of the estimation of the expected symptom in response to the neurostimulation.
